(19) United States
(12) Patent Application Publication
(10) Pub. No.: US 2014/0271584 A1
(43) Pub. Date: Sep. 18, 2014

(54) Methods and Compositions for Direct Reprogramming of Somatic Cells to Stem Cells, and Uses of these Cells

(71) Applicant: THE RESEARCH FOUNDATION FOR THE STATE UNIVERSITY OF NEW YORK, ALBANY, NY (US)

(72) Inventors: Yupo MA, East Setauket, NY (US); Wenbin LIAO, Port Jefferson, NY (US)

(73) Assignee: THE RESEARCH FOUNDATION FOR THE STATE UNIVERSITY OF NEW YORK, ALBANY, NY (US)

(21) Appl. No.: 14/212,712

(22) Filed: Mar. 14, 2014

Related U.S. Application Data
(60) Provisional application No. 61/789,232, filed on Mar. 15, 2013.

(51) Int. Cl. C12N 5/074; A61K 35/12; C12N 5/0797

(52) U.S. Cl. CPC C12N 5/0696; C12N 5/0623; A61K 35/12
USPC 424/93.21; 435/377; 435/467; 424/93.7

(57) ABSTRACT

Presented herein are methods of generating an induced stem cell (iSC) from a somatic cell, by contacting the somatic cell with an induction factor that reprograms the somatic cell to generate an iSC. The induction factor can be a genetic construct or a fusion protein. Where the induction factor is a genetic construct, the construct bears one or more nucleotide sequences encoding one or more reprogramming elements selected from OCT4, SOX2, NANOG, and a Notch pathway molecule, or an active fragment or derivative thereof. The genetic construct can have a lentiviral or episomal vector backbone. The induction factor can also be a fusion protein, with the reprogramming element being a protein selected from OCT4, SOX2, NANOG, or a Notch pathway molecule, or an active fragment or derivative thereof. The fusion protein can be TAT protein or an active fragment or derivative thereof.

### BACKGROUND OF THE DISCLOSURE

Stem cells are undifferentiated cells that have extensive proliferation potential, can differentiate into several cell lineages, and repopulate tissues upon transplantation. Stem cells can give rise to more progenitor cells having the ability to generate a large number of mother cells that can in turn give rise to differentiated, or differentiable daughter cells. The quintessential stem cell is the embryonic stem cell, as it has unlimited self-renewal and pluripotent differentiation potential (Orkin, Int. J. Dev. Biol. 42:927-34, 1998; Reubinoff et al., Nat Biotech. 18:399404, 2000; Shamblott et al., Proc. Natl. Acad. Sci. U.S.A. 95:13726-31, 1998; Thomson et al., Science 282:114-7, 1998; Thomson et al., Proc. Natl. Acad. Sci. USA. 92:7844-8, 1995; Williams et al., Nature 336:684-7, 1988). Embryonic stem (ES) cells have been extensively studied for use in providing sources of new tissue. However, in addition to the ethical and supply issues surrounding the use of human fetal tissue as a source of ES cells, ES cells have other challenges, including genetic instability and cancer risk.

To address ethical and other issues related to ES cells, somatic cells have recently been reported to reprogram into pluripotent cells, termed induced pluripotent stem (iPS) cells, using a combination of defined transcription factors. The reprogramming of somatic cells to iPS cells is a new area of signicant potential. These cells have great therapeutic potential because they can be tailored specifically to a patient or disease. In principle, an individual suffering from a genetic, degenerative, or malignant disorder could submit a biopsy for reprogramming to an iPS cell. Following reprogramming, a prescribed course of iPS cell differentiation to a specific tissue type could be initiated that would allow one to cure a given disorder. Proof of principle experiments have been done in mouse models. For example, mice displaying a phenotype similar to human sickle cell anemia were cured of the disease through somatic cell reprogramming and directed differentiation into blood cell progenitor populations. This is a clear demonstration of potential therapeutic uses for iPS cells.

These iPS cells, to date, are quite similar to embryonic stem cells and have the same pluripotent characteristics. ES/iPS cells have the capacity to self-renew and differentiate into all cell types. However, while experiments with stem cell technologies show great promise, major hurdles remain to be overcome before induced cell technology can be considered safe for human treatment.

iPS cells, like embryonic stem (ES) cells, have numerous challenges, including genetic instability and cancer risk. In one instance, activation of exogenously-introduced iPS-inducing genes may lead to the malignant transformation of iPSs (for example, when oncogenic transcription factors, such as c-Myc, are used). In addition, lentiviral or retroviral delivery could possibly cause a random insertion of the inducing gene into the genome and it is feasible that this delivery could happen within the coding sequence of a vital gene, thus disrupting the gene and causing a damaging mutation leading to developmental or malignant disorders.

To move away from ES/iPS cells because of the cancer risk, transdifferentiation, a process of reprogramming a cell directly from one mature cell type to another cell type, has been reported. The mature cells derived from direct reprogramming are likely insufficient for cellular therapy due to their limited capacity to self-renew and regenerate. Despite this, direct reprogramming of somatic cells into multipotent or lineage-restricted stem cells is highly desired because such cells could have adequate capacity of self-renewal and differential potential, yet have reduced tumorigenic potential.

Recently, several published research accounts have reported the direct reprogramming of somatic skin cells to NSCs using a lentiviral vector expressing SOX2 or a combination of defined transcriptional factors However, the described process requires co-culturing the somatic cells with feeder cells, which carries additional risks. Finally, the efficiency of direct reprogramming of such cells is very low, resulting in insufficient numbers for clinical use. The safety issues and low efficiency of direct reprogramming are barriers for clinical applications of these cells.

Thus, there remain numerous barriers to be solved before these promising therapies are ready for use in human subjects.

### BRIEF SUMMARY OF THE DISCLOSURE

Presented herein are methods of generating an induced stem cell (iSC) from a somatic cell, by contacting the somatic cell with an induction factor that reprograms the somatic cell to generate an iSC. The induction factor includes at least one “reprogramming element”, that is, an element that directs the somatic cell to de-differentiate, and an “expression-enabling element”, which enables entry and/or expression of the reprogramming element within the somatic cell. The induction factor can be a genetic construct or a fusion protein.

Where the induction factor is a genetic construct, the construct bears one or more nucleotide sequences encoding one or more reprogramming elements selected from OCT4, SOX2, NANOG, and a Notch pathway molecule, or an active fragment or derivative thereof. The construct may encode multiple reprogramming elements, or only a single reprogramming element. The single reprogramming element can encode one of OCT4, SOX2, or NANOG. Alternatively, the construct can include two reprogramming elements, selected from OCT4 and SOX2, or OCT4 and NANOG, or SOX2 and NANOG. The construct may further comprise any combination of two or more reprogramming elements, selected from OCT4, SOX2, NANOG, and a Notch pathway molecule. The expression-enabling element of the genetic construct can be a lentiviral or episomal vector backbone.

The induction factor can also be a fusion protein, with the reprogramming element being a protein selected from OCT4, SOX2, NANOG, or a Notch pathway molecule, or an active fragment or derivative thereof. The expression-enabling element of the fusion protein can be TAT protein or an active fragment or derivative thereof.

The somatic cell can reprogrammed by the steps of: (i) contacting the somatic cell with the induction factor under conditions and for a time sufficient for the induction factor to induce the somatic cell to de-differentiate; and (ii) culturing the de-differentiated somatic cell under conditions and for a time sufficient to reprogram the de-differentiated somatic cell to generate an iSC.

Using the disclosed methods, the somatic cell is cultured in step (ii) with stem cell induction media, and can be cultured in steps (i) and (ii) in the absence of feeder cells. In one example, the stem cell induction media can be a human neural stem cell media.

The somatic cell can be selected from, for example, an amniotic fluid cell, a bone marrow cell, a blood cell, a myocardial cell, a dermal or epidermal cell, a pancreatic cell,a fat cell or a fibroblast. The iSC generated by the methods disclosed herein can be a neural stem cell, bone stem cell, bone marrow stem cell, lung stem cell, kidney stem cell, endothelial stem cell, myocardial stem cell, muscle stem cell, mesenchymal stem cell, hepatic stem cell, pancreatic stem cell, dermal stem cell, epidermal stem cell, or hematopoietic stem cell.

This disclosure further encompasses an induced stem cell (iSC) produced by the methods described herein. In one example, the iSC is an induced neural stem cell (iNSC). In another example, the iSC is an induced endothelial stem cell.

This disclosure also presents methods of repairing or regenerating a tissue in a subject, involving administering an induced stem cell (iSC) generated according to the disclosed methods to a subject in need of tissue repair or regeneration. For example, disclosed methods can be administered to a subject to treat myocardial infarction, congestive heart failure, stroke, ischemia, peripheral vascular disease, alcoholic liver disease, cirrhosis, Parkinson's disease, Alzheimer's disease, diabetes, cancer, arthritis, a wound, immunodeficiency, anemia, or a genetic disorder.

### BRIEF DESCRIPTION OF THE FIGURES

FIGS. 1A-1D. Reprogramming of AF cells into iNSC by OCT4. (A), WT AF cells; (B), GFP-AF cells; (C), Day 4 of AF cells after OCT4 infection; (D), OCT4-AF cells replated in a monolayer. OCT4 transduced AF cells show morphology of typical NSC cells (C) as early as 3 days after transfer to NSC medium as compared to GFP control (B).

FIGS. 2A-2C. (A), OCT4 AF-iNSCs are able to form neurospheres in a petri dish. (B, C), AF-iNSCs are positive for Nestin immunostaining

FIG. 3. Heatmap of expression levels of 23 neural stem cell genes in OCT4 AF-iNSCs (AF-iNSC1 and AF-iNSC2), hcx NSC and control AF cells.

FIGS. 4A-4C. OCT4 AF-iNSCs differentiate into neuron, astrocyte and oligodendrocyte as shown by Tuj1 (A), GFAP (B), or CNPase (C) staining

FIG. 5. OCT4 AF-iNSCs survive (A) and differentiate (B) in the mouse brain one month after transplantation.

FIG. 6. After reprogramming, SOX2 over-expressed AF cells show a dramatic change in morphology which resembles the morphology of the hcx NSC line and differs from GFP or WT control cells.

FIG. 7. SOX2 AF-iNSCs are able to form neurospheres in a petri dish, which could not be seen in HFF after ectopic SOX2 expression.

FIGS. 8A-8B. SOX2 AF-iNSCs are positive for Nestin (A) and Musashi-1 (B) immunostaining

FIG. 9. SOX2 AF-iNPCs are able to differentiate into neurons, astrocytes, and oligodendrocytes as shown by MAP2, GFAP or CNPase immunostaining

FIGS. 10A-10C. Sustained K+ currents were recorded in response to applying voltage to neurons differentiated from SOX2 AF-iNSCs. (A), Sox2-F; (B), positive control, hcx NSC cell line; (C), negative control, GFP-AF.

FIGS. 11A-11B. Engraftment of SOX2 AF-iNSCs in vivo. (A), the transplanted AF-iNSC engraft in the hippocampus of recipient mouse brain as shown by post-transplantation brain fluorescence. (B), the engrafted cells are able to differentiate into neural cells positive for the neuron specific marker Tuj1. DAPI was used to identify nuclei.

FIGS. 12A-12D. Generation of SOX2 AF-iNSCs by a non-integrating vector. (A), transduced cells with GFP-only vector (pCEP-GFP control) maintain AF cell-type morphology. Inset to (A), GFP expression in transduced cells. (B), After culture in ReNcell neural stem cell medium, pCEP-50X2 transducted AF cells show typical NSC-like morphology. (C), A human NSC line. (D), GFP control.

FIGS. 13A-13B. TAT-50X2-6×His protein was expressed overnight in BL21 E. coli at 25° C. In the presence of 0.2 mM IPTG and 0.5% glucose. Soluble protein was incubated with Ni-NTA (Qiagen) beads in the presence of 10 mM imidazole, washed, and eluted with buffer containing 250 mM imidazole. (A), Coomassie stain; B-Western blot. Lane 1—Uninduced bacteria; 2—Induced, lysate; 3—Cleared lysate; 4—Flowthrough; 5—Wash 1; 6-9—Elution fractions 1-4. (B), Western blot with primary antibody: Mouse mAb anti-SOX2 (EMD Millipore, Billerica, Mass.), 1:1000. Secondary—anti-mouse-HRP (EMD Millipore, Billerica, Mass.) 1:5000.

FIGS. 14A-14C. Generation of iNSCs from BM-MSC. (A) SOX2 transduced BM-MSC form colony clusters as compared to control. (B) The colonies are positive for Nestin immunostaining (C) Dissociated cells from colonies form neurospheres.

FIGS. 15A-15F. FLK1-positive colonies were picked up, cultured and passaged. (A, B, E), Passage1 (P1). (C, D, F), Passage 2 (P2). (E) and (F) are live stains with antibodies against FLK1 showing most cells are FLK1-positive.

FIGS. 16A-16B. Ac-LDL uptake (A) and tube formation on Matrigel (B) of induced endothelial cells.

FIGS. 17A-17F. In vitro (A) and in vivo (B) tube/vessel formation of induced endothelial cells. Cells in in-vivo Matigel implant also express CD31 (C-F).

FIG. 18. GFP labeled induced FLK1 positive cells engraft and differentiate into CD31 positive cells in SCID mice liver.

### DETAILED DESCRIPTION OF THE DISCLOSURE

This disclosure provides methods of generating a multipotent stem cell from a somatic cell. The inventors have developed methods to generate these multipotent stem cells by contacting the somatic cells with at least one “induction factor” that alters the somatic cell and induces the somatic cell to become the desired stem cell.

The inventors have managed to overcome several barriers to clinical applications of stem cell technologies in humans. First, the inventors have overcome the barrier present in reprogramming a somatic cell with multiple exogenous genes, by identifying and showing successful reduction to practice of the induction of stem cells from somatic cells with several individual genes that, acting alone, become a “master switch” for stem cell induction. Second, the inventors have overcome the inability to generate sufficient numbers of induced stem cells, by identifying methods of stem cell induction that work in many somatic cell types, including somatic cells that are plentiful and/or reproduce rapidly, thus generating large numbers of induced stem cells sufficient for transplantation to humans. In addition, the inventors have overcome the requirement for generation of induced stem cells by co-culturing somatic cells with feeder cells, by identifying methods that generate iSCs without the need for feeder cell co-culture. Further, the inventors have overcome the potential issues involved in unpredictable integration of a genetic vector into a host chromosome, by presenting specialized induction factors, including episomal vectors and fusion proteins, that generate induced stem cells without requiring integration into the host genome.

The term “stem cell” as used herein refers to an immature cell that is capable of differentiating into a number of final, differentiated cell types. A stem cell may divide symmetrically, to form two daughter stem cells, or asymmetrically, to form a daughter stem cell and a somatic cell. Characteristics of stem cells include loss of contact inhibition, anchorage independent growth, de novo expression of alkaline phosphatase and/or activation of the germ line specific Oct4 promoter. Oct4, a member of the Pou domain, class 5, transcription factors (Pou 5fl) (Genbank Accession No. S68053) is one of the mammalian POU transcription factors expressed by early embryo cells and germ cells, and is a marker for stem cells in mammals.

Stem cells may be totipotent, multipotent/pluripotent, or unipotent cells. Totipotent stem cells typically have the capacity to develop into any cell type and are usually embryonic in origin. Multipotent or pluripotent (the terms are used interchangeably herein) stem cells are typically capable of differentiating into several different, final differentiated cell types. Unipotent cells are typically capable of differentiating into a single cell type. Non-embryonic stem cells are usually multipotent or unipotent. Multipotent stem cells are considered to be “lineage-restricted”, meaning that these stem cells can give rise to a cell committed to forming a particular limited range of differentiated cell types.

The primary cell lineages are endoderm cells, which include liver, intestine, pancreas, lung, and other internal organs; ectoderm cells, which include skin, hair, and neuronal cells; and mesoderm cells, which include hematopoietic, blood, muscle, cardiovascular, and bone cells. However, the primary lineages can be further restricted, for example, hematopoietic cells can be further restricted to myeloid or lymphoid lineages.

The term “progenitor” as used herein, refers to a cell that is committed to a particular cell lineage and which gives rise to cells of this lineage by a series of cell divisions. A progenitor cell can more differentiated than a stem cell but is not itself fully differentiated. An example of a progenitor cell would be a myoblast, which is capable of differentiation to only one type of cell, but is itself not fully mature or fully differentiated.

The term “differentiation” as used herein, refers to a developmental process whereby cells become specialized for a particular function, for example, where cells acquire one or more morphological characteristics and/or functions different from that of the initial cell type. The term “differentiation” includes both lineage commitment and differentiation of a cell into a mature, fully differentiated cell. Differentiation may assessed, for example, by monitoring the presence or absence of lineage markers, using immunohistochemistry or other procedures known to a worker skilled in the art. Differentiated progeny cells derived from progenitor cells may be, but are not necessarily, related to the same germ layer or tissue as the source tissue of the progenitor cells.

An “induced stem cell” is a stem cell that is produced or generated from a somatic cell, by reprogramming the somatic cell to alter its state of differentiation to become a stem cell. The term “somatic cell” includes any cell that is not itself a gamete, germ cell, gametocyte, or undifferentiated stem cell. Somatic cells are typically more differentiated than stem cells; thus, somatic cells must be reprogrammed to de-differentiate (that is, to become less differentiated and acquire one or more characteristics of a stem cell). A somatic cell is “reprogrammed” (directed to de-differentiate into a stem cell) according to the methods disclosed herein by contacting the somatic cell with an induction factor that alters the somatic cell's developmental program. Somatic cells can also be reprogrammed to generate an induced progenitor cell. The induced lineage-restricted stem/progenitor cells bear capacity of self-renewal and differential potential.

Specific stem/precursor cells that can be induced by the disclosed methods include neural stem cells, bone marrow stem cells, lung stem cells, kidney stem cells, endothelial stem cells, myocardial stem cells, muscle stem cells, bone cells, mesenchymal stem cells, hepatic stem cells, pancreatic stem cells, dermal stem cells, epidermal stem cells, and hematopoietic stem cells.

For example, somatic cells can be reprogrammed to generate neural stem cells (NSCs). It has been thought that the subventricular zone of the lateral ventricles and the dentate gyms of the hippocampus are the main sources of human adult NSCs, which are considered to be a reservoir of new neural cells. Adult NSCs with potential neural capacity have also been isolated from white matter and inferior prefrontal subcortex in the human brain. Several references in stem cell biology have raised promising possibilities of replacing lost/damaged or degenerative neural cells by stem cell transplantation. However, sources of NSCs, sufficient quantities, and control of the differentiations for clinical uses represent a major barrier for transplantation. Thus, the generation of NSCs from non-brain sources has great therapeutic potential for treatment of various neural disorders.

The “induction factor” described herein has the ability to direct, reprogram, or induce the somatic cell to become a stem cell.

The induction factor includes at least a reprogramming element, which directs the development of the somatic cell away from its normal course, and an expression-enabling element, which enables entry and expression of the reprogramming element in the somatic cell. The reprogramming element is any gene, protein, or active fragment or derivative thereof of a gene or protein, that directs the somatic cell to become a stem cell. The induction factor can be a genetic construct, with one or more genetic reprogramming and expression-enabling elements. Alternatively, the induction factor can be a fusion protein, with reprogramming and expression-enabling elements comprised of polypeptides.

An active fragment is a fragment of the reprogramming element which is capable of directing de-differentiation of a somatic cell into a stem cell. An active fragment would include the active region or functional domain, for example, an active fragment of a transcription factor would contain at least one or both of a DNA-binding domain and a co-factor binding site, while an active fragment of a ligand would contain at least a receptor binding/activation domain, and an active fragment of a receptor would contain at least one or both of an intracellular signaling domain and a ligand-binding domain. A derivative of the reprogramming element or the active fragment thereof is the protein or active fragment thereof which includes some modification, mutation, or addition, for example, including another chemical substance (such as polyethylene glycol), or which is associated with mutation such as addition, deletion, insertion or substitution of at least one, and preferably one to several amino acids. In other words, derivatives of a reprogramming element, and active fragments thereof, include mutants, modified forms, and modification products of the reprogramming element, and active fragments thereof, that are capable of directing de-differentiation of a somatic cell into a stem cell.

The reprogramming element can be one or more of OCT4, SOX2, NANOG, or a Notch pathway molecule. Notch pathway molecules include Notch receptors (Notch1-4) and ligands of the Delta-Serrate-Lag (DSL) type (Jag1, Jag2, and delta-like 1/Dll1, Dll3 and Dll4), as well as transcription factor C promoter-binding factor (CBF1), also known as recombination signal binding protein for immunoglobulin kappa J region (RBPJ-κ) or kappa-binding factor 2 (KBF2). Expression of any one or more of these reprogramming elements within the somatic cell directs de-differentiation of the somatic cell into a stem cell. The stem cell type ultimately induced depends on the induction media used to generate the stem cell, as described in greater detail below.

OCT4 (octamer-binding transcription factor 4), also known as POU5F1 (POU domain, class 5, transcription factor 1), OCT3, or OTF3, is encoded by the POU5F1 gene. Human OCT4 has at least two to five splice variant isoforms. As an example, the sequence for a specific human OCT4 variant, POU domain, class 5, transcription factor 1 isoform 1, is set forth in UniProtKB/Swiss-Prot Database Accession No. Q01860. Specific examples of the nucleotide and amino acid sequences of OCT4 are provided as SEQ ID NO: 1 and SEQ ID NO: 2, respectively. In one embodiment, an induction factor contains OCT4 as the sole reprogramming element.

SOX2, or SRY (sex determining region Y)-box 2, is a transcription factor that is essential for maintaining self-renewal of stem cells. Sox2 is a member of the Sox family of transcription factors, which have been shown to play key roles in many stages of mammalian development. The inventors have determined that SOX2, like OCT4, is a “master switch” that can reprogram somatic cells in the absence of other reprogramming elements. As an example, the sequence for a specific human SOX2 nucleotide sequence is provided as SEQ ID NO: 3. As another example, the amino acid sequence of SOX2 is provided as SEQ ID NO: 4. In one embodiment, an induction factor contains SOX2 as the sole reprogramming element. In a further embodiment, an induction factor contains both OCT4 and SOX2 as reprogramming elements.

Other reprogramming factors include NANOG, a homeobox transcription factor involved in stem cell proliferation and self-renewal. The NANOG nucleotide sequence is available as NCBI Reference Sequence: NM_{—}024865.2. The NANOG amino acid sequence is available as NCBI Reference Sequence: NP_{—}079141.2. In one embodiment, an induction factor contains SOX2 as the sole reprogramming element. In a further embodiment, an induction factor contains NANOG with either OCT4 or SOX2 as reprogramming elements. In a further embodiment, an induction factor contains NANOG with both OCT4 and SOX2 as reprogramming elements.

Other reprogramming factors include signaling molecules of the Notch pathway, such as Notch1-4, Jag1, Jag2, Dll1, Dll3, Dll4, and CBF1/RBPJ-κ/KBF2.

In one embodiment of the induction factor, the induction factor is a genetic construct. In this embodiment, the reprogramming element is a nucleic acid sequence encoding either the entirety of the reprogramming element, or an active fragment or derivative thereof, and the expression-enabling element is a genetic vector.

Functional derivatives and homologs of the reprogramming factors disclosed herein are further contemplated for use in the disclosed methods. As used herein, a “functional derivative” is a molecule which possesses the capacity to perform the biological function of a molecule disclosed herein. For example, a functional derivative of a reprogramming factor as disclosed herein is a molecule that is able to functionally substitute for a reprogramming factors, e.g., in the reprogramming of AF cells to iSCs. Functional derivatives include fragments, parts, portions, equivalents, analogs, mutants, mimetics from natural, synthetic or recombinant sources including fusion proteins. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. Additions to amino acid sequences include fusions with other peptides, polypeptides or proteins.

A variant of a molecule is meant to refer to a molecule substantially similar in structure and function to either the entire molecule, or to a fragment thereof. Thus, as the term variant is used herein, two molecules are variants of one another if they possess a similar activity even if the structure of one of the molecules is not found in the other, or if the sequence of amino acid residues is not identical. The term variant includes, for example, splice variants or isoforms of a gene. Equivalents should be understood to include reference to molecules which can act as a functional analog or agonist. Equivalents may not necessarily be derived from the subject molecule but may share certain conformational similarities. Equivalents also include peptide mimics.

A “homolog” is a protein related to a second protein by descent from a common ancestral DNA sequence. A member of the same protein family (for example, the OCT family or SOX family) can be a homolog. A “functional homolog” is a related protein or fragment thereof that is capable of performing the biological activity of the desired gene, i.e, is able to functionally substitute for the disclosed reprogramming factors in the reprogramming of somatic cells to pluripotent or multipotent cells. Homologs and functional homologs contemplated herein include, but are not limited to, proteins derived from different species.

An OCT4 functional derivative or homolog can have 75%, 80%, 85%, 90%, 95% or greater amino acid sequence identity to a known OCT4 amino acid sequence, or 75%, 80%, 85%, 90%, 95% or greater amino acid sequence identity to a OCT4 family member or variant thereof. An OCT4 functional derivative or homolog can have, for example, 75%, 80%, 85%, 90%, 95% or greater amino acid sequence identity to SEQ ID NO: 1.

A SOX2 functional derivative or homolog can have 75%, 80%, 85%, 90%, 95% or greater amino acid sequence identity to a known SOX2 amino acid sequence, or 75%, 80%, 85%, 90%, 95% or greater amino acid sequence identity to a SOX2 family member or variant thereof. A SOX2 functional derivative or homolog can have, for example, 75%, 80%, 85%, 90%, 95% or greater amino acid sequence identity to NCBI Reference Sequence: NP_{—}003097.1.

The expression-enabling element enables entry and expression of the reprogramming element in the somatic cell. An expression-enabling element can be integrative, meaning it directs the reprogramming element to integrate into the somatic cell genome, or non-integrative, meaning it enables expression of the reprogramming element from an extrachromosomal location. In either case, the expression-enabling element is typically provided as a backbone vector into which the nucleic acid sequence for the reprogramming factor is cloned by techniques known in the art.

Integrative vectors include retrovirus, lentivirus, adenovirus, adeno-associated virus, and other vectors that, once introduced into a cell, integrate into a chromosomal location within the genome of the subject and provide stable, long-term expression of the reprogramming factor. Exemplary vectors for stem cell induction are described, for example, in Yu J, et al., Science 318:1917-20 (2007) and Hanna J, et al., Cell 133:250-64 (2008). The nucleotide sequence of one or more reprogramming elements can be cloned into the vector sequence, the vector is grown in appropriate host cells, and used to reprogram the somatic cell using the methods described in greater detail below.

Non-integrative vectors include episomal vectors, as well as engineered lentivirus vector variants that are non-integrative. These vectors direct expression of the reprogramming element as a separate genetic element. Because these vectors do not integrate into the chromosome, the risk of integration into a gene resulting in genetic harm or inactivation is avoided. The absence of chromosomal integration means that episomal vectors are more easily lost from the somatic cell; however, once the somatic cell is reprogrammed into a stem cell and delivered to a subject, the induced stem cell will be directed to re-differentiate within the tissue of the subject, and accordingly the vector is no longer needed.

Episomal vectors can be generated from, for example, BKV (BK polyoma virus), BPV-1 (bovine papillomavirus type 1), Epstein-Barr virus (EBV)-plasmid, EBV-BAC (bacterial artificial chromosome), EBNA-1 (Epstein-Barr nuclear antigen 1), scaffold matrix attachment region (S/MAR)-plasmid, S/MAR-BAC, Minichromosome, or human artificial chromosome (HAC)-based vectors. In a specific example, the episomal vector is pCEP, an episomal expression vector that uses a promoter, such as the simian virus 40 early promoter (SV40), cytomegalovirus immediate-early promoter (CMV), Ubiquitin C promoter (UBC), human elongation factor 1α promoter (EF1A), phosphoglycerate kinase 1 promoter (PGK), and spleen focus-forming virus (SFFV) promoter. The vector also contains a multiple cloning site for introduction of the sequence of the reprogramming factor or factors, an EBV replication origin, and an EBNA-1 nuclear antigen, to permit extrachromosomal replication and expression in mammalian cells. References for episomal reprogramming of somatic cells are described, for example, in Meng X, et al, Mol Ther. 20:408-16 (2012); Okita K, et al., Stem Cells 31:458-66 (2013); and Yu J, et al., PLoS One 6:e17557 (2011).

The vector for expressing a reprogramming factor comprises a promoter operably linked to the reprogramming factor gene. The phrase “operably linked” or “under transcriptional control” as used herein means that the promoter is in the correct location and orientation in relation to a polynucleotide to control the initiation of transcription by RNA polymerase and expression of the polynucleotide. In a preferred embodiment, the reprogramming factor is operably linked to a strong promoter, such as the human elongation factor 1α promoter (EF1A), or the spleen focus-forming virus (SFFV) promoter.

In another embodiment of the induction factor, the induction factor is a fusion protein. In this embodiment, the reprogramming element is a polypeptide including either the entirety of the reprogramming element protein, or an active fragment or derivative thereof, and the expression-enabling element is a polypeptide that mediates entry of the fusion protein into the somatic cell interior, wherein the reprogramming element directs de-differentiation of the cell.

The expression-enabling element can be a fusion protein comprising, for example: a protein transduction domain of a known cell penetrating peptide (CPP) such as penetratin or Tat protein; a chimeric CPP or derivative thereof, such as transportan, a stearylated-transportan derivative, or an MPG protein transduction domain; a synthetic CPP, such as a poly-arginine or “oligoarginine” region comprising between 6 and 12 arginine repeats; and “second generation” CPPs such as a repeating R-Ahx-R motif (reviewed in Said H F, et al., Cell Mol. Life Sci. 67:715-26 (2010).

Exemplary CPP constructs are provided in Table 1 as follows:

| Name | Origin/design | Sequence |
|---|---|---|
| Penetratin | Antennapedia | RQIKIWFQNRRMKWK (SEQ ID NO 5) |
| (pAntp) | | |
| Tat 48-60 | HIV-1 transactivator (Tat) | GRKKRRQRRRPPQ (SEQ ID NO 6) |
| Transportan | Galanin + Mastoparan | GWTLNSAGYLLGKINLKALAALAKKI |
| | | L (SEQ ID NO 7) |
| MPG | HIV-1 gp 41 + NLS SV40 | GALFLGFLGAAGSTMGAWSQPKKKR |
| | | KV (SEQ ID NO 8) |
| MAP | Model amphipatic peptide | KLALKLALKALKAALKA (SEQ ID NO |
| | | 9) |

In a specific example, the fusion protein comprises TAT protein, or an active fragment such as the sequence GRKKRRQRRRPPQ (SEQ ID NO: 6), fused to the reprogramming element. For fusion protein embodiments, typically a single reprogramming element, or active fragment or derivative thereof will be used, for example, one of OCT4, SOX2, or NANOG; however, fusion proteins that include more than one reprogramming element, or active fragments or derivatives thereof, are also contemplated by the inventors. Once the fusion protein is introduced onto the surface of the somatic cell, the expression-enabling element enables entry of the fusion protein into the cell, where the reprogramming element can then begin direction of the cell to de-differentiate into a stem cell.

In one embodiment, the inventors have developed methods to generate multipotent stem cells by contacting amniotic fluid cells with OCT4 alone. The generated multipotent stem cells express FLK1 and are capable of propagating or maintaining in vitro in the ES cell medium or EGM-2 medium. In a specific embodiment, the generated multipotent stem cells do not express human embryonic stem cell markers, SSEA3 and Tra-1-60, and do not form a teratoma when injected to SCID mice. These generated multipotent stem cells also have the ability of differentiation to endothelial cells, which bear properties including: (1) taking up acetylated-low density lipoprotein (Ac-LDL); expressing CD31; (2) tubular formations in vitro or vivo; and (3) engraftment in the liver.

Accordingly, methods to reprogram a somatic cell using the induction factor are as follows.

Somatic cells can be obtained from a biological sample. Sources of somatic cells that can be used to generate the desired stem cell include amniotic fluid (AF), bone marrow (BM), adipose tissue, blood, plasma, epidermal tissue, placenta, or any organ or tissue. Somatic cells can originate from various tissue or organ systems, including, but not limited to, blood, nerve, muscle, skin, gut, bone, kidney, liver, pancreas, thymus, and the like. In one example, the somatic cell is an AF or BM cell.

AF cells are found in the AF that surrounds the fetus in the womb. AF can be extracted through the mother's abdomen using a needle in a process called amniocentesis, which can be used to test for genetic diseases in utero. As amniocentesis typically does not affect the pregnancy, and is a routine procedure, use of AF cells avoids both ethical and supply concerns related to ES cells. Amniotic fluid contains fetal cells sloughed off by the developing fetus. These fetal cells are usually negative for markers of: the hematopoietic lineage (CD45⁻), hematopoietic stem cells (CD34⁻, CD1331⁻) and endothelial cells (CD31⁻, FLK-1⁻, CD144³¹ ). Amniotic fluid can contain several cell types, including germ cells of mesodermal, endodermal, and ectodermal germ layers; placental cells; amniotic epithelial cells; trophoblasts; and amniotic fluid stem cells. Due to the ability of the transforming factors disclosed herein to reprogram any cell to induce the desired stem cell, even a heterologous population of cells can be essentially uniformly induced to generate a single iSC type. Therefore, although AF and other biological samples can be further purified to obtain a single somatic cell type, according to the methods presented herein they do not need to be a pure population prior to inducing the desired stem cells.

If desired, different cell types can be fractionated into subpopulations. This may be accomplished using standard techniques for cell separation including, but not limited to, enzymatic treatment; cloning and selection of specific cell types, including but not limited to selection based on morphological and/or biochemical markers; selective growth of desired cells (positive selection), selective destruction of unwanted cells (negative selection); separation based upon differential cell agglutinability in the mixed population as, for example, with soybean agglutinin; freeze-thaw procedures; differential adherence properties of the cells in the mixed population; filtration; conventional and zonal centrifugation; centrifugal elutriation (counter-streaming centrifugation); unit gravity separation; countercurrent distribution; electrophoresis; fluorescence activated cell sorting (FACS); and the like.

Identifying the characteristics of a cell population can be performed upon or following isolation of a sample or expansion of somatic cells, prior to reprogramming. Alternatively, or in addition, cell typing can be performed after reprogramming, to determine the characteristics of the iSCs generated from reprogramming. Cells can be characterized by, for example, by growth characteristics (e.g., population doubling capability, doubling time, passages to senescence), karyotype analysis (e.g., normal karyotype; maternal or neonatal lineage), flow cytometry (e.g., FACS analysis), immunohistochemistry and/or immunocytochemistry (e.g., for detection of epitopes), gene expression profiling (e.g., gene chip arrays; polymerase chain reaction (for example, reverse transcriptase PCR, real time PCR, and conventional PCR)), protein arrays, protein secretion (e.g., by plasma clotting assay or analysis of PDC-conditioned medium, for example, by Enzyme Linked ImmunoSorbent Assay (ELISA)), mixed lymphocyte reaction (e.g., as measure of stimulation of PBMCs), and/or other methods known in the art.

Isolated cells, or untreated samples such as AF, can be used to initiate cell cultures. Cells or samples are transferred to sterile tissue culture vessels either uncoated or coated with extracellular matrix or ligands such as laminin, collagen (native, denatured or crosslinked), gelatin, fibronectin, or other extracellular matrix proteins. Cells are cultured in any culture medium capable of sustaining growth of the cells such as, but not limited to, DMEM (high or low glucose), advanced DMEM, DMEM/MCDB 201, Eagle's basal medium, Ham's F10 medium (F10), Ham's F-12 medium (F12), Hayflick's Medium, Iscove's modified Dulbecco's medium, Mesenchymal Stem Cell Growth Medium (MSCGM), DMEM/F12, RPMI 1640, and CELL-GRO-FREE (Corning cellgro, Corning, N.Y.). The culture medium can be supplemented with one or more components including, for example fetal bovine serum, preferably about 2-15% (v/v); equine serum; human serum; fetal calf serum; beta-mercaptoethanol, preferably about 0.001% (v/v); one or more growth factors, for example, platelet-derived growth factor (PDGF), epidermal growth factor (EGF), fibroblast growth factor (FGF), vascular endothelial growth factor (VEGF), insulin-like growth factor-1 (IGF-1), leukocyte inhibitory factor (LIF) and erythropoietin; amino acids, including L-valine; and one or more antibiotic and/or antimycotic agents to control microbial contamination, such as, for example, penicillin G, streptomycin sulfate, amphotericin B, gentamicin, and nystatin, either alone or in combination.

The somatic cells can be cultured to expand the cell numbers, prior to reprogramming. Sufficient numbers of somatic cells may be isolated in the initial sample; however, even if an acceptable number of somatic cells is present in the initial sample, expansion of the cells in culture can provide an even greater supply of somatic cells for reprogramming. Methods of culturing and expanding somatic cells are known in the art. See, for example, Helgason et al., Basic Cell Culture Protocols, 4th Edition, Human Press Publishing, 2013; and Mitry et al, Human Cell Culture Protocols, 3rd Edition, Human Press Publishing, 2012.

Once sufficient numbers of somatic cells are generated, the somatic cells are seeded onto issue culture plates are seeded with somatic cells, in the range of 5,000 to 25,000 cells per cm². In a specific example, 10,000 to 20,000 cells per cm² are seeded onto a tissue culture plate or flask that is coated with laminin, collagen, gelatin, fibronectin, or other extracellular matrix proteins.

As a first step in the reprogramming process, the somatic cells are contacted with the induction factor, for a sufficient time and under conditions that allow the induction factor to gain entry into the somatic cells and reprogram them to de-differentiate. Sufficient time can be 1 hour to 1 week, or 2, 4, 6, 8, 10, 12 hrs, or 1 to 3 days. Conditions depend in part on the induction factor used. Exemplary conditions for reprogramming are disclosed in the following references:

Integrative vector culture conditions: see, e.g., Yu J, et al., Science 318:1917-20 (2007) and Hanna J, et al., Cell 133:250-64 (2008).

Non-integrative/episomal culture conditions: see, e.g., Meng X, et al, Mol Ther. 20:408-16 (2012); Okita K, et al., Stem Cells 31:458-66 (2013); and Yu J, et al., PLoS One 6:e17557 (2011).

Fusion protein culture conditions: see, e.g., Zhang H, et al., Biomaterials 33:5047-55 (2012); and Tang Y, et al., Cell Reprogram. 13:99-112 (2011).

Expression of recombinant OCT4 and SOX2 proteins. OCT4, SOX2, and other reprogramming factors can be expressed in E. coli (using, for example, the pET28 expression plasmid; Novagen), in insect cells (using the Sf9-baculovirus system; Invitrogen), in yeast, or in mammalian cells. In mammalian cells (Chinese hamster ovary), the protein can be expressed (using an expression plasmid such as the pFUSE-h1gG1-Fc2 plasmid; Invivogen) along with a signal sequence, such as IL2 signal sequence, which allows the protein to be secreted from the cell into the culture media. In bacteria and Sf9 cells, the recombinant proteins are expressed with a 6×His fusion tag for purification, while the Fc region (CH2 and CH3 domains) of the human IgG1 heavy chain and the hinge region are used as the fusion tag in mammalian cell expression.

Cells are induced to lineage-restricted stem/progenitor cells under a tissue or cell type specialized medium. The method would be used to direct reprogramming by overexpression of at least one reprogramming factor, to generate lineage specialized stem cells such as neural stem cells, skin stem cells, liver stem cell, pancreatic stem cells, bone marrow stem cells, lung stem cells, heart stem cells, kidney stem cells, endothelial stem cells, and mesenchymal stem cells. As an example, AF cells can be transduced with OCT4 for two or three days and the transduced cells are then placed in a neural stem cell medium to induce OCT4 expressing cells into neural stem/progenitor cells.

One benefit of the methods disclosed herein is that the stem cells can be generated from culture of somatic cells in a “feeder free” system; that is, the somatic cells can be cultured to generate stem cells in the absence of a feeder cell layer.

Feeder cell layers are adherent, growth-arrested but viable cells that are cultured to form a bottom layer on which other cells are grown in a co-culture system. Feeder cell layers provide an extracellular matrix and secrete known and unknown factors into the medium. Many mammalian cell types, such as stem cells, will not survive or proliferate without physical contact with a feeder layer. As such, feeder cells, typically mouse or human fibroblasts, are often required in stem cell culture methods. However, the presence of feeder cells is a detriment to establishing clinical grade stem cells, which for use in humans must be produced without any animal cells or products. The methods provided herein allow stem cell generation without the use of feeder cells.

Following contact with the induction factor, the de-differentiated somatic cell is cultured in specialized medium and conditions designed to produce the desired stem cell. Examples of media and growth conditions that can be utilized to produce specific iSCs are as follows:

Generation of cardiomyocyte iSCs: Chamuleau S A, et al., Cardiovasc Res. 82(3):385-7 (2009).

Generation of hepatic iSCs: Liu J, et al, Sci Rep. 3:1185 (2013); Hirose Y, et al, Exp Cell Res. 315(15):2648-57 (2009).

Generation of pancreatic iSCs: Kordes C, et al, PLoS One. 7(12):e51878 (2012); Moshtagh P R, et al, J Physiol Biochem. 2012.

Generation of endodermal and intestinal iSCs: Kim T H, et al., Proc Natl Acad Sci USA. 109(10):3932-7 (2012).

Generation of dermal/epidermal iSCs: Chen F, et al., Cytotechnology. 2013.

Generation of neural/photoreceptor iSCs: Ballios B G, et al., Biol Open. 1(3):237-46 (2012).

In a particular example, AF cells are reprogrammed to generate induced neural stem cells (iNSCs). Somatic cells treated with induction factors as described above can be initially maintained in AF cell growth medium, for example, for 1 day to 2 weeks. Once induction of neural stem cells is desired, the AF medium is changed to a neural stem cell (NSC) medium to induce neural stem cell formation. Exemplary NSC medium is a defined medium, such as DMEM/F12, with supplements including 1, 2, 3, 4, 5, 6, 8, 10, 12, or all of: L-glutamine, human serum albumin, human transferrin, putrescine dihydrochloride, human recombinant insulin, L-thyroxine, tri-iodo-thyronine, progesterone, sodium selenite, heparin, corticosterone, basic fibroblast growth factor (bFGF or FGF2), epidermal growth factor (EGF), and/or antibiotics. Although DMEM/F12 commonly contains HEPES (hydroxyethyl piperazineethanesulfonic acid) as a buffering agent, HEPES is preferably absent from the NSC medium and thus DMEM/F12 minus HEPES is preferably used as the base medium. An exemplary NSC medium is ReNcell medium (EMD Millipore, Billerica, Mass.), which is a DMEM/F12 medium minus HEPES with L-glutamine, human serum albumin, human transferrin, putrescine dihydrochloride, human recombinant insulin, L-thyroxine, tri-iodo-thyronine, progesterone, sodium selenite, heparin, and corticosterone, and which the inventors further supplemented with 20 ng/ml human FGF-2 and 20 ng/ml human EGF. NSC medium can be changed every 1-3 days, preferably every day.

AF induced NSCs (AF-iNSC) form within 2 to 3 days after culture is NSC medium, as can be evidenced by the formation of cell clusters. These clustering cells can form neurospheres when transferred to low attachment surfaces such as an uncoated tissue culture vessel.

To expand cell cultures of AF-iNSCs, the cells can be treated with a proteolytic or detachment enzyme suitable for stem cells, such as ACCUTASE (Innovative Cell Technologies, Inc., San Diego, Calif.), and passaged to laminin coated tissue culture plates after 6-10 days. Following the initial passage, the AF-iNSC can then be passaged every 5 days until the cells reach approximately 80-90% confluence.

In another specific example, AF cells are reprogrammed to generate induced multipotent stem cells (iMSCs) by OCT4 overexpression. After transduction with a lentivirus driving expression of OCT4 under control of the SFFV or EFla promoter, cells can be transferred to human ES (embryonic stem cell) medium, such as DMEM/F12, or mTESR1/mTESR2 (StemCell Technologies). Where serum-free ES media is used, the media should be supplemented with a defined, serum-free serum substitute containing, for example, bovine serum albumin, transferrin, and/or insulin, which serum substitute is able to grow and maintain undifferentiated ES cells in culture. Examples of suitable serum substitutes include Knockout Serum replacement (Life Technologies), and serum replacement media available from Sigma-Aldrich, which are added to the base media (e.g., DMEM/F12 or similar media) in amounts of 10-30%, preferably 15-25%, or as recommended by the manufacturer. ES media is preferably additionally supplemented with 1-5 mM, preferable 1-3 mM glutamine; 0.01-1 mM, preferably 0.05-0.15 mM, non-essential amino acids; 1-20 ng/ml, preferably 8-12 ng/ml, bFGF; and 50-150 μM, preferably 80-120 μM, β-Mercaptoethanol.

To improve reprogramming, additional compounds may be added to the culture media. The inventors have found that addition of a TGF-beta receptor (TGF-βR) inhibitor, such as the TGF-β1R inhibitor II known as “616452” or a functional derivative thereof, and/or 8-Br-cAMP or a functional derivative thereof. 1-20 μM, preferably 5-15 μM, even more preferably 8-12 μM, of the TGF-PR inhibitor may be added. 0.01-1 mM, preferably 0.05-0.15 mM, even more preferably 0.08-0.12 mM 8-Br-cAMP may also or alternatively be added.

616452 is 2-(3-(6-Methylpyridin-2-yl)-1H-pyrazol-4-yl)-1,5-naphthyridine with structure:

[chemical structure]

8-Br-cAMP, also known as 8-Bromoadenosine 3′,5′-cyclic monophosphate, is (1S,6R,8R,9R)-8-(6-amino-8-bromopurin-9-yl)-3-hydroxy-3-oxo-2,4,7-trioxa-3λ5-phosphabicyclo[4.3.0]nonan-9-ol, with the structure:

[chemical structure]

To create endothelial stem cells, transduced cells or iMSCs can be transferred to an endothelial cell medium, such as EGM2 (Lonza, Inc.) or ENDOGROW (EMD Millipore). In a particularly preferred embodiment, transduced cells are cultured in EGM-2 media (Lonza, Inc.), optionally with 2-(3-(6-Methylpyridin-2-yl)-1H-pyrazol-4-yl)-1,5-naphthyridine or a functional derivative thereof, and 8-Br-cAMP or a functional derivative thereof, also added to the media. The inventors have determined that transduced cells cultured after transduction under these or similar conditions are reliably reprogrammed to induced multipotent stem cells (iMSCs). iMSCs generated under these conditions are FLK1 positive (FLK1⁺), and negative for the embryonic stem cell markers SSEA3 and TRA-1-60 (SSEA3⁻, TRA-1-60⁻).

FLK1 is also known as Fetal Liver Kinase-1 (FLK-1), Vascular Endothelial Growth Factor Receptor-2 (VEGFR-2), CD309, or Kinase Insert Domain Receptor (KDR). The UniProt Accession number for FLK is P35968. FLK1+ cells are considered to be vascular progenitor cells that define the vascular and hematovascular lineages, capable of differentiating into endothelial cells, pericytes, vascular smooth muscle cells, hematopoietic cells, and cardiac cells. SSEA3 (Stage-specific embryonic antigen 3, also known as SSEA-3) and TRA-1-60 are markers for pluripotent and embryonic stem cells.

These FLK1 iMSCs can differentiate into functional somatic cells of the brain, liver, skin, heart, kidney, pancreas, gall bladder, intestine, skeletal muscle and lung in an appropriate medium, such as any media disclosed above.

The inventors have also found that FLK1 positive cells can differentiate to CD31⁺ endothelial cells when cultured in endothelial cell media. These induced endothelial cells (iECs) can form tube-like structures in vivo and in vitro (e.g., on Matrigel or similar three-dimensional matrices), and can take up or scavenge acetylated-low density lipoprotein (Ac-LDL) in vitro. These abilities are characteristic of endothelial cells. Furthermore, iECs can survive and proliferate when transplanted in vivo. Thus, iECs generated by the methods disclosed herein are characterized by one or more of: ability to take up/scavenge acetylated-low density lipoprotein (Ac-LDL); ability to form tubes or tube-like structures in vitro and/or in vivo; expression of CD31-positivity; and engraftment in vascular structures or organs, such as the liver. The ECs generated by these methods are useful for the treatment of vascular disorders, by administration or transplantation of these cells to an individual in need of treatment for a vascular disorder. Vascular disorders include atherosclerosis, peripheral vascular disease, post-angioplasty restenosis, pulmonary arterial hypertension, vein-graft disease or graft-versus-host disease (GvHD), arterial aneurysms, allergic angiitis and granulomatosis (Churg-Strauss disease), Behget's syndrome, Cogan's syndrome, Henoch-Schonlein purpura, Kawaski disease, leukocytoclastic vasculitis, polyarteritis nodosa (PAN), microscopic polyangiitis, polyangiitis overlap syndrome, Takayasu's arteritis, temporal arteritis, transplant rejection, Wegener's granulomatosis, and thromboangiitis obliterans (Buerger's disease).

The present invention further provides a method for repairing or regenerating a tissue or differentiated cell lineage in a subject. The method involves obtaining an iSC from a somatic cell and administering the iSC to a subject, e.g., a subject having a myocardial infarction, congestive heart failure, stroke, ischemia, alcoholic liver disease, cirrhosis, Parkinson's disease, Alzheimer's disease, diabetes, cancer, arthritis, an internal or external wound, immunodeficiency, anemia including aplastic anemia, or a genetic disorder, or other diseases or conditions where an increase or replacement of a particular cell type/tissue, or cellular re-differentiation is desirable.

The iSCs can be used for autologous (i.e., cells are obtained from the same subject to be treated with the reprogrammed stem cells), allogeneic (i.e., cells are obtained from another subject of the same species as the subject to be treated), or xenogeneic (i.e., cells are obtained from a subject of a different species from the subject to be treated) transplantation.

Some non-limiting examples of damage that can be repaired and reversed by the invention include surgical removal of any portion (or all) of the diseased or damaged organ or tissue, drug-induced damage, toxin-induced damage, radiation-induced damage, environmental exposure-induced damage, sonic damage, heat damage, hypoxic damage, oxidation damage, viral damage, age or senescence-related damage, inflammation-induced damage, immune cell-induced damage, for example, transplant rejection, immune complex-induced damage, and the like.

As used herein, the terms “subject” and “patient” are used interchangeably and refer to an animal, including mammals such as non-primates (e.g., cows, pigs, horses, cats, dogs, rats etc.) and primates (e.g., monkey and human).

The terms “treatment”, “treating”, and the like, as used herein include amelioration or elimination of a developed disease or condition once it has been established or alleviation of the characteristic symptoms of such disease or condition. As used herein these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of iSCs to a patient that is not at the time of administration afflicted with the disease or condition. “Preventing” also encompasses preventing the recurrence or relapse-prevention of a disease or condition or of symptoms associated therewith, for instance after a period of improvement.

The cells can be administered as a pharmaceutical/therapeutic cell composition that comprises a pharmaceutically-acceptable carrier and iSCs as described and exemplified herein. In one example, therapeutic cell compositions can comprise AF cells induced to differentiate along a neural pathway or lineage. The therapeutic cell compositions can comprise cells or cell products that stimulate cells in the patient's tissue requiring regeneration to divide, differentiate, or both. It is preferred that the therapeutic cell composition induce, facilitate, or sustain repair and/or regeneration of the damaged or diseased tissues or organs in the patient to which they are administered.

The cells can be administered to the patient by injection. For example, the cells can be injected directly into the damaged tissue of the patient, or can be injected onto the surface of the tissue, into an adjacent area, or even to a more remote area with subsequent migration to the patient's tissue requiring regeneration or repair. In some preferred aspects, the cells can home to the diseased or damaged area.

The cells can also be administered in the form of a device such as a matrix-cell complex. Matrices include biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. The cells of the invention can also be seeded onto three-dimensional matrices, such as scaffolds and implanted in vivo, where the seeded cells may proliferate on or in the framework, or help to establish replacement tissue in vivo with or without cooperation of other cells. Also contemplated are matrix-cell complexes in which the cells are growing in close association with the matrix and when used therapeutically, growth, repair, and/or regeneration of the patient's own damaged tissue is stimulated and supported, and proper angiogenesis is similarly stimulated or supported. The matrix-cell compositions can be introduced into a patient's body in any way known in the art, including but not limited to implantation, injection, surgical attachment, transplantation with other tissue, and the like.

A successful treatment could thus comprise treatment of a patient with a disease, pathology, or trauma to a body part with a therapeutic cell composition comprising iSCs, in the presence or absence of another cell type. For example, and not by way of limitation, the cells preferably at least partially integrate, multiply, or survive in the patient. In other preferred embodiments, the patient experiences benefits from the therapy, for example from the ability of the cells to support the growth of other cells, including stem cells or progenitor cells present in the damaged or diseased tissue, from the tissue in-growth or vascularization of the tissue, and from the presence of beneficial cellular factors, chemokines, cytokines and the like, but the cells do not integrate or multiply in the patient. In some aspects, the patient benefits from the therapeutic treatment with the cells, but the cells do not survive for a prolonged period in the patient. For example, in one embodiment, the cells gradually decline in number, viability or biochemical activity. In other embodiments, the decline in cells may be preceded by a period of activity, for example growth, division, or biochemical activity.

The administering is preferably in vivo by transplanting, implanting, injecting, fusing, delivering via catheter, or providing as a matrix-cell complex, or any other means known in the art for providing cell therapy.

The present disclosure is further illustrated by the following non-limiting examples. The contents of all references cited herein are incorporated by reference in their entirety.

### EXAMPLES

### Cell Cultures

Amniotic fluid (AF) cell isolation—Human second trimester AF was obtained by ultrasound-guided amniocentesis performed on pregnant women for routine prenatal diagnosis purposes. All human samples were obtained after the approval from the Ethical Review Board of the Stony Brook University Hospital and the informed consent from the subjects. 5 ml fluids were washed with PBS and centrifuged at 350 g at 4° C. for 10 min. The pellets were plated in T25 tissue culture flasks and grown in AF culture medium. AF culture medium is DMEM/F12 (Gibco/Life Technologies, Inc.) containing 15% heat-inactivated fetal bovine serum (Hyclone/Thermo Scientific), 10 ng/ml human bFGF (Peprotech, Rocky Hill, N.J.), and 100 U/ml penicillin/streptomycin (Gibco/Life Technologies, Inc.). Medium was changed on day 3 by removal of the non-adherent cells in the supernatant. The medium was refreshed every 2-3 days. At 10-12 days after plating, the cells were trypsinized and passaged routinely at 80-90% confluence.

Human Foreskin Fibroblasts (HFF) were purchased from the American Type Tissue Collection (ATCC) and maintained in DMEM with 10% FBS. Cells from a human cortex neural stem cell line (cx NSC) were purchased from EMD Millipore, Billerica, Mass. and maintained according to manufacturer's instruction.

### Tranduction of AF Cells by Lentivirus Infection, Lipofection or TAT-Protein

Promoters for lentiviral and episomal plasmids were replaced with the spleen focus forming virus (SFFV) promoter sequence (SEQ ID NO: 10) or the EF1α (human elongation factor 1 alpha) promoter sequence (SEQ ID NO: 11).

Transduction via integrating plasmid. Lentivirus based vectors carrying human SOX2 or OCT4 gene, or GFP only gene, each expressed under control of either the SFFV promoter or EF1α promoter, were packaged using the 293FT cell line (Invitrogen/Life Technologies, Inc.) to produce lentivirus. The viruses were concentrated by centrifugation and stored at −80° C. One day before infection, tissue culture dishes were coated with poly-L-ornithine (PLO) and laminin AF, BM, or HFF cells were seeded at a density of 15,000 cells/cm². Lentiviruses were added at a MOI of 10-100 in the presence of 8 μg/ml polybrene (EMD Millipore, Billerica, Mass.) for 6 hours. Efficiency was measured after 24-48 hours by expression of green fluorescence.

Transduction via non-integrating plasmid. The pCEP-SOX2 plasmid, a non-integrating EBNA1-based episomal vector, was used to transiently express SOX2 in AF cells using the LIPOFECTAMINE 2000 transfection agent (Invitrogen/Life Technologies, Inc.).

Transduction via recombinant protein expression. Purified TAT-SOX2 recombinant protein, obtained from a constructed TAT-SOX2 vector and expressed in B21 bacteria, was used to treat AF cells.

### Reprogramming to Neural Stem Cells

The AF, BM, or HFF cells that were treated by lentiviruses, non-integrating plasmids or TAT fusion proteins were maintained in AF cell growth medium as described above for 2 days and switched into human neural stem cells (NSC) medium: ReNcell medium (EMD Millipore, Billerica, Mass.) supplemented with 20 ng/ml human FGF-2 and 20 ng/ml human EGF (Peprotech, Rocky Hill, N.J.) on day 3. NSC medium was changed daily. The cells were treated with accutase and passaged to laminin coated tissue culture plates on day 7-9. The AF induced NSC (AF-iNSC) were then passaged every 5 days when the cells reached 80-90% confluence. For neurosphere formation, cells were plated in a low adherent (uncoated) petri dish with NSC medium.

### Differentiation of AF-iNSC

For random differentiation and maturation into three neural cell lineages, AF-iNSCs or BM-iNSCs were cultured in PLO/Laminin coated glass coverslips in ReNcell medium without bFGF and EGF for 14 days. For specific differentiation, iNSC cells were induced by addition of 20 ng/ml BDNF (brain derived neurotrophic factor) and 20 ng/ml GDNF (Glial cell-derived neurotrophic factor) (Peprotech, Rocky Hill, N.J.).

### Immunostaining

Cells were fixed in 4% paraformaldehyde for 15 minutes at room temperature and washed with PBS. Nonspecific antibody binding was blocked using 1% BSA for 30 minutes, and cells were permeabilized with 0.3% Triton X-100 (Sigma) in PBS (PBS-T) for 30 minutes at room temperature. Cells were rinsed and then incubated in primary antibody containing 0.1% overnight at 4° C. After washing in PBS, cells were incubated in secondary antibody 1 hour at room temperature. Cells were immunostained with the following anti-human primary antibodies: anti-Nestin, anti-βIII tubulin (Tuj1), anti-MAP2 anti-glial fibrillary acidic protein (GFAP), Musashi-1, or CNPase (2′,3′-cyclic nucleotide 3′-phosphodiesterase, an oligodendrocyte-specific enzyme). Primary antibodies were detected with the PE (phycoerythrin) conjugated secondary antibody. Stained cells were preserved in anti-fading mount solution that contained DAPI. Stained cells were examined and photographed using an EVOS fluorescent microscope (Life Technologies, Inc.).

### Heatmap of NSC Wxpression

Total RNAs of AF induced NSCs, AF cells and hcx NSCs were extracted using ALLPREP DNA/RNA Mini Kit (Qiagen) and cDNA was synthesized using QuantiTect Rev. Transcription Kit (Qiagen). RNA quantity and quality (2100 Bioanalyzer, Agilent Technologies) was determined to be optimal before further processing. The Affymetrix Human HG-U133plus2 GeneChip arrays hybridization, staining, and scanning, were performed using Affymetrix standard protocols (Affymetrix, Santa Clara, Calif.) as previously described in Stony Brook University DNA Microarray Core Facility. All genes of neurogenesis and hematopoiesis according to Gene ontology (GO) terms (AmiGO, available online at the Geneontology website) are analyzed and the upregulation or downregulation fold changes were normalized to AF cells. The heat-map of gene expression levels was generated by R software.

### Electrophysiology

Glass coverslips containing differentiated cells derived from AF-iNSC cells or a human cortex neural stem cell line (hcx NSC; EMD Millipore, Billerica, Mass.) were transferred to a Zeiss microscope with DIC and phase-contrast optics. In the whole-cell patch clamp, cells with a relatively large cell body and neurite like structures were chosen for recording. Cells were perfused with a standard bathing medium (140 mM NaCl, 5 mM KCl, 1.5 mM CaCl2, 1 mM MgCl2, 10 mM HEPES, pH 7.2, 37 1C). Electrodes were pulled from borosilicate glass and filled with intracellular recording solution (100 mM KCH3SO3, 40 mM KCl, 0.2 mM EGTA, 0.02 mM CaCl2, 1 mM MgCl2, 2 mM ATP, 300 mM GTP, 10 mM HEPES buffer) for voltage clamp measurements. Potassium currents were elicited by stepwise depolarization of the membrane in 20-mV increments from −110 to +110 mV.

### In Vivo Transplantation

iNSCs were dissociated by Accutase and resuspended in PBS. Eight to ten-week old NOD/SCID mice (Jackson Lab) were anesthetized with ketamine/xylazine (100 mg/kg and 10 mg/kg, respectively). For transplantation, 1-2 μL of cell suspension (60,000 cells) were delivered to the hippocampus in the right hemisphere through stereotaxic surgery. The coordinate of the injection site was posterior: 1.7 mm; lateral: 1.5 mm ventral: 2.0 mm rostral/caudal, using the position of the bregma as reference. One month after transplantation, mouse brain were prefixed by intracardial perfusion and dehydrated in 20% sucrose for cryostat sectioning. Brain sections were examined under a fluorescent microscope and immunostained with a neural cell antibody.

### Results

### EXAMPLE 1

### OCT4 or SOX2 Overexpression Via Lentiviral Expression De-Differentiates AF Cells

AF cells could be easily cultured and expanded in large amount from only 5 ml AF sample. The average doubling time of these cells is 12±1.6 hours. Passage 3 to 5 AF cells were plated in PLO/LN (poly-L-ornithine or poly-L-lysine) coated tissue culture plates and infected with Oct4 or Sox2 lentivirus particles at MOI of 10-100 in the presence of 8 μg/ml polybrene for 6 hours. 24-48 hours after infection, the efficiency of lentiviral infection was determined under fluorescent microscope by GFP expression. Typically the percentage of GFP positive cells can reach higher than 80%.

Transduced cells were plated to assess growth. Numerous colonies were present only 7 or 9 days post-transduction. The OCT4 transduced colonies in particular were able to expand and continue to grow over 10 passages. The inventors found that addition of small chemical compounds such as TGF-βreceptor I inhibitor II (10 uM) and 8-Br-cAMP (0.1 mM) enhanced the reprogramming efficiency by 10-15%. The induced colonies were ALK1 positive but negative for ESC markers, such as SSEA-3 and Tra-1-60.

### EXAMPLE 2

### OCT4 or SOX2 Overexpression via Lentiviral Transduction can Reprogram AF Cells to Neural Precursor Cells

After OCT4- or SOX2-induced dedifferentiation, cells were transferred to ReNcell medium plus bFGF and EGF. On Day 4 in ReNcell medium, OCT4 and SOX2-induced AF cells formed cell clusters in culture (FIG. 1C; FIG. 7, “AF”) and these clusters could be dissociated into single cells for monolayer culture. The induced cells resembled neural stem cells in morphology (FIG. 1 D; FIG. 6 “SOX2”), including development of long, thin processes which are drastically different from the WT or GFP-only control cells (FIGS. 1A-B; FIG. 6 “GFP” and “WT”).

Neurosphere formation is a feature of NSC growth in vitro. Not surprisingly, the induced NSC from AF cells could form typical neurospheres in low attachment dish (FIG. 2A; FIG. 7 “AF”).

Next, the inventors used antibodies to Nestin and Mushashi-1, which are neural stem cell markers, to detect the expression of NSC-specific markers in the induced NSCs (AF-iNSCs). In neurosphere and monolayer culture systems, the induced cells were positive for Nestin (FIGS. 2B-C; FIG. 8A) and Musashi-1 (FIG. 8B).

AF-iNSCs are similar to human cxNSC in gene expression pattern. A gene expression array was conducted to compare the similarity between OCT4 AF-iNSCs and a human NSC line. The results showed that NSC genes including DCLK1 (doublecortin-like kinase 1), MSX2 (muscle segment homeobox 2), and TFAP2C (transcription factor AP-2 gamma), are upregulated in AF-iNPCs as compared to control AF cells, and in human cx NSCs (FIG. 3).

AF-iNSCs can differentiate into three lineages in vitro. AF-iNSCs were cultured on PLO/LN coated glass coverslips for differentiation, after 2 weeks in differentiation medium depleted of growth factors bFGF and EGF. As shown in FIGS. 4A-C and FIG. 9, the inventors found that AF-iNSCs could mature into neurons, astrocytes and oligodendrocytes, which are the three lineages of cells in the neural system, as identified by immunostaining for Tuj-1 (beta III Tubulin, a neuronal marker), MAP2 (microtubule-associated protein 2, a neuronal marker), GFAP (glial fibrillary acidic protein, an astrocyte marker) and CNPase (2′,3′-Cyclic-nucleotide 3′-phosphodiesterase, an oligodendrocyte marker). Electrophysiological analysis was also used to characterize the neural cells after differentiation. Currents in the cells could be recorded in a whole-cell patch clamp (FIG. 10A), which was similar to the neural cells differentiated from the human CX NSC line (FIG. 10B), while in GFP-only control cells, only baseline currents were detected (FIG. 10C).

Integration of AF-iNPCs into animal brain. To test whether AF-iNPCs are able to differentiate and incorporate into neural tissue in vivo, the inventors injected 60,000 Passage 3 AF-iNPCs into the striatum of NOD/SCID mouse brain. Animal brain sections were obtained 1 month post-transplantation. The inventors identified injected cells by GFP fluorescence. By using lineage-specific antibody staining, the inventors found that AF-iNPCs were capable of differentiating into mature neural cells in mouse brain. Regarding the risk of tumorigenesis using reprogrammed cells such as iPS derived cells in vivo, the inventors injected AF-iNPCs subcutaneously or intracerebrally to detect their tumorigenic potentials. Animal brain sections were obtained 2 weeks or 1 month post-transplantation. In both time points, the inventors observed injected cells as shown by GFP fluorescence (FIG. 5A; FIG. 11A). By using lineage-specific antibody staining, AF-iNSCs were shown to be capable of differentiating into mature cells in the mouse brain (FIG. 5B; FIG. 11B). The results showed no tumor or teratoma formation 3 months after injection.

### EXAMPLE 3

### OCT4 Overexpression via Lentiviral Transduction can Reprogram AF Cells to Endothelial Stem Cells

After viral transduction, cells were transferred to human ES (embryonic stem cell) medium (supplemented with 20% Knockout Serum replacement, 2 mM glutamine, 0.1 mM non-essential amino acids, 10 ng/ml bFGF, 100 μM β-Mercaptoethanol) or endothelial cell medium, such as EGM2 (Lonza, Inc.). Numerous colonies appeared on day 9 that were not seen in the AF cells transduced with control lentiviruses expressing GFP. More than 90% of colonies were FLK1 positive by day 11.

In order to increase the colony forming efficiency, the inventors tried different culture media and found that the addition of TGF-beta receptor1 inhibitor II, 616452 at 10 μM or 8-Br-cAMP at 0.1 mM in the culture media accelerated and increased the efficiency of colony formation by approximately 15%.

616452 is 2-(3-(6-Methylpyridin-2-yl)-1H-pyrazol-4-yl)-1,5-naphthyridine with structure:

[chemical structure]

8-Br-cAMP, also known as 8-Bromoadenosine 3′,5′-cyclic monophosphate, is (1S,6R,8R,9R)-8-(6-amino-8-bromopurin-9-yl)-3-hydroxy-3-oxo-2,4,7-trioxa-3λ5-phosphabicyclo[4.3.0]nonan-9-ol, with the structure:

[chemical structure]

The colonies formed in the ES medium were prone to detach from the plate despite still proliferating, when maintained in ES medium. In contrast, when cells were switched to EGM2 medium on day 6, the colonies were adherent throughout the culturing process. The inventors isolated these colonies and pooled them for expansion after dissociation. They were able to form new colonies with more than 90% FLK1 positive (FIG. 15A-F). The inventors also used EGM2 for the reprogramming from the beginning with the addition of TGF-beta receptor inhibitor 616452 and 8-Br-cAMP. Although no 3-dimensonal colonies were observed, there were monolayer colonies which were also positive for FLK-1.

FLK1 positive cells can differentiate to endothelial cells. The induced endothelial cells were able to form tube-like structures on Matrigel and uptake acetylated-low density lipoprotein (Ac-LDL) in vitro (FIG. 16A-B). Furthermore, blood vessels were observed in cell-matrigel mixed subcutaneous transplants in NOD/SCID mice 3 weeks after injection (FIG. 17A-F). The inventors also transfected these induced cells with lentiviruses expressing GFP. The resulting GFP labeled cells were transplanted to the liver of irradiated mice through intra portal vein injection. One month later, the GFP positive donor cells could be found around the central vein area. A proportion of the engrafted cells were also expressing CD31, indicative of the transplanted cells differentiating into endothelial cells in vivo (FIG. 18).

To further characterize induced FLK1 positive cells, the inventors enriched this population by sorting FLK1 positive cells. The FLK-1 (+) cells continued to grow and formed colonies but the number of FLK-1 expressing cells gradually decreased during culture (p1:>90%, p2: 77% ,p6: 20%). The sorted cells could be expanded for at least 14 passages.

### EXAMPLE 4

### SOX2 Overexpression via Episomal Transduction can Reprogram AF Cells to Neural Precursor Cells

For potential clinical applications, transgene-free AF-iNSCs are highly desired in order to prevent potential adverse effects due to lentiviral integration or to the interference of residual expression of reprogramming factors on the differentiation of iNSCs. The inventors used an EBNA1-based episomal vector, which has high transfection efficiency and non-integrating features. The inventors tested if an episomal vector expressing SOX2, pCEP-SOX2 was able to generate AF-iNSCs. By lipofection, the efficiency of gene transduction was ˜50% at 24 hours as seen in pCEP-GFP control vector (FIG. 12A). Ten days after transduction by pCEP-SOX2, AF cells showed a similar morphological change to that seen in AF cells after lentiviral infection (FIG. 12B) similar to hcx NSCs (FIG. 12C).

### EXAMPLE 5

### Generation of AF-iNSCs using Recombinant TAT-SOX2 Protein

The ideal method to generate clinically applicable AF-iNSCs is using protein induction. The inventors successfully purified TAT-SOX2 from E. coli (FIG. 13A-B) and AF cells exposed to a TAT-SOX2 protein exhibited a NSC-like morphology change and form neurospheres in vitro.

### EXAMPLE 6

### Generation of iSCs from Bone Marrow Cells

Bone marrow-derived MSCs (BM-MSCs) are widely used in preclinical or clinical investigation for cell therapy, due to their advantage in autologous application. The inventors ectopically expressed SOX2 in BM-MSC with lentivirus. At 10 days after infection and in a NSC medium, neurosphere-like colonies could be seen in SOX2 induced cells, which were absent in GFP control cells (FIG. 14A). The colonies were also positive for Nestin immunostaining (FIG. 14B). In addition, the cells from the colonies were able to form neurospheres in a petri dish (FIG. 14C).

The present invention provides methods that show direct reprogramming of somatic cells such as amniotic fluid (AF) and bone marrow (BM) cells, as non-limiting examples, to iNSCs and endothelial cells using a single transcriptional factor, either Sox2 or OCT4. The methods would be applied to direct reprogramming of somatic cells, including for example AF cells, to other types of lineage restricted stem cells. A small number of 50,000 to 100,000 amniotic cells are able to directly reprogram to more than a billion iNSCs within a matter of weeks. The multipotent or lineage-restricted stem cells generated through direct reprogramming of somatic cells would have the potential to be used as a source for both allogeneic and autologous therapy in different disorders such as genetic and degenerative diseases.

What is claimed is:

1. A method of generating an induced stem cell (iSC) from a somatic cell, comprising the steps of: (i) contacting said somatic cell with an induction factor that reprograms the somatic cell to de-differentiate; and (ii) culturing said de-differentiated somatic cell under conditions and for a time sufficient to reprogram said de-differentiated somatic cell to generate an iSC.

2. The method of claim 1, wherein the induction factor is a genetic construct comprising one or more nucleotide sequences encoding one or more reprogramming elements selected from OCT4, SOX2, NANOG, and a Notch pathway molecule, or an active fragment or derivative thereof.

3. The method of claim 2, wherein the genetic construct comprises a lentiviral or episomal vector backbone.

4. The method of claim 3, wherein the genetic construct encodes a single reprogramming element.

5. The method of claim 4, wherein the single reprogramming element is one of OCT4 or SOX2, or an active fragment or derivative thereof.

6. The method of claim 4, wherein expression of the reprogramming element is under control of the spleen focus forming virus (SFFV) promoter or the human elongation factor 1α (EF) promoter.

7. The method of claim 6, wherein the single reprogramming element is OCT4 or an active fragment or derivative thereof.

8. The method of claim 1, wherein the induction factor is a fusion protein comprising a protein selected from OCT4, SOX2, NANOG, or a Notch pathway molecule, or an active fragment or derivative thereof.

9. The method of claim 7, wherein the fusion protein comprises TAT protein or an active fragment or derivative thereof.

10. The method of claim 1, wherein said somatic cell is cultured in steps (i) and (ii) in the absence of feeder cells.

11. The method of claims 1, wherein the somatic cell is cultured in step (ii) with stem cell induction media.

12. The method of claim 11, wherein the stem cell induction media comprises human neural stem cell media.

13. The method of claim 1, wherein the somatic cell is selected from an amniotic fluid cell, a bone marrow cell, a blood cell, a myocardial cell, a dermal or epidermal cell, a pancreatic cell, or a fibroblast.

14. The method of claim 1 or 13, wherein the iSC generated is a neural stem cell, bone stem cell, bone marrow stem cell, lung stem cell, kidney stem cell, endothelial stem cell, myocardial stem cell, muscle stem cell, mesenchymal stem cell, hepatic stem cell, pancreatic stem cell, dermal stem cell, epidermal stem cell, or hematopoietic stem cell.

15. An induced stem cell (iSC) produced by the method of claim 1.

16. The iSC of claim 15, wherein the iSC is a neural stem cell or an endothelial stem cell.

17. A method of repairing or regenerating a tissue in a subject, comprising administering an induced stem cell (iSC) generated according to the method of claim 1 to a subject in need of tissue repair or regeneration.

18. The method of claim 17, wherein the subject has myocardial infarction, congestive heart failure, stroke, ischemia, peripheral vascular disease, alcoholic liver disease, cirrhosis, Parkinson's disease, Alzheimer's disease, diabetes, cancer, arthritis, a wound, immunodeficiency, anemia, or a genetic disorder.


SEQUENCE LISTING (11 sequences)

SEQ ID NO: 1 - DNA, 1080 bp, Homo sapiens
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggtgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaac

SEQ ID NO: 2 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - DNA, 2513 bp, Homo sapiens
ggatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaa

SEQ ID NO: 4 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - protein, 15 aa, Drosophila melanogaster
RQIKIWFQNRRMKWK

SEQ ID NO: 6 - protein, 13 aa, Human immunodeficiency virus
GRKKRRQRRRPPQ

SEQ ID NO: 7 - protein, 27 aa, Artificial Sequence
  synthesized peptide
GWTLNSAGYLLGKINLKALAALAKKIL

SEQ ID NO: 8 - protein, 27 aa, Artificial Sequence
  synthesized peptide
GALFLGFLGAAGSTMGAWSQPKKKRKV

SEQ ID NO: 9 - protein, 17 aa, Artificial Sequence
  synthesized peptide
KLALKLALKALKAALKA

SEQ ID NO: 10 - DNA, 423 bp, Spleen focus-forming virus
agctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggtacc

SEQ ID NO: 11 - DNA, 1178 bp, Homo sapiens
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga